(19) United States
(12) Patent Application Publication
(10) Pub. No.: US 2015/0185042 A1
(43) Pub. Date: Jul. 2, 2015

(54) DYNAMIC COMPUTATION OF DISTANCE OF TRAVEL ON WEARABLE DEVICES

(71) Applicants: Stuart Crawford, Piedmont, CA (US); Dean Achelis, San Francisco, CA (US); Max Everett Utter, II, San Francisco, CA (US)

(72) Inventors: Stuart Crawford, Piedmont, CA (US); Dean Achelis, San Francisco, CA (US); Max Everett Utter, II, San Francisco, CA (US)

(73) Assignee: AliphCom, San Francisco, CA (US)

(21) Appl. No.: 14/144,494

(22) Filed: Dec. 30, 2013

(51) Int. Cl. G01C 22/00; A61B 5/00

(52) U.S. Cl. CPC G01C 22/00; A61B 5/6801

(57) ABSTRACT

Techniques for dynamic computation of distance of travel on wearable devices are described. Disclosed are techniques for receiving motion data over context windows from one or more sensors coupled to a wearable device, determining a number of motion units of each context window, determining a motion unit length of each context window as a function of the number of motion units of each context window and a duration of each context window, determining a distance of travel of each context window, and determining a total distance of travel over all context windows. The motion unit length of each context window is variable from the motion unit length of another context window. In some embodiments, the total distance of travel is presented on an interface coupled to the wearable device.

### FIELD

Various embodiments relate generally to wearable electrical and electronic hardware, computer software, human-computing interfaces, wired and wireless network communications, telecommunications, data processing, and computing devices. More specifically, disclosed are techniques for dynamically computing the distance of travel of a user of a wearable device.

### BACKGROUND

With the advent of computing devices in smaller personal and/or portable form factors and an increasing number of applications (i.e., computer and Internet software or programs) for different uses, devices for detecting the number of steps taken and/or the distance traveled are becoming more popular. At least one drawback of the conventional techniques is that data is usually poorly captured using conventional devices.

Conventional devices for detecting the distance of travel typically do not take into account a broad array of factors that may affect the result. Further, conventional devices do not generally permit the user to improve the way for determining the distance traveled. Further, conventional devices do not generally verify whether their determination of the distance traveled is accurate.

Thus, what is needed is a solution for dynamically computing distance of travel without the limitations of conventional techniques.

### BRIEF DESCRIPTION OF THE DRAWINGS

Various embodiments or examples (“examples”) are disclosed in the following detailed description and the accompanying drawings:

FIG. 1 illustrates an exemplary wearable device with a dynamic distance manager in some applications, according to some examples;

FIG. 2 illustrates an application architecture for an exemplary wearable device with a dynamic distance manager, according to some examples;

FIG. 3 illustrates another application architecture for an exemplary wearable device with a dynamic distance manager, according to some examples;

FIG. 4A illustrates exemplary motion data for an activity for use by an exemplary wearable device with a dynamic distance manager, according to some examples;

FIG. 4B illustrates exemplary motion data for another activity for use by an exemplary wearable device with a dynamic distance manager, according to some examples;

FIG. 5 illustrates a network for use by a plurality of exemplary wearable devices with a dynamic distance manager, according to some examples;

FIG. 6 illustrates an exemplary decision tree for use by an exemplary wearable device with a dynamic distance manager, according to some examples;

FIG. 7 illustrates exemplary applications of a dynamic distance manager, according to some examples;

FIG. 8 illustrates exemplary motion data for use in creating an exemplary model for use by a dynamic distance manager, according to some examples;

FIG. 9 illustrates exemplary motion data for use in adjusting or calibrating an exemplary model for use by a dynamic distance manager, according to some examples;

FIG. 10A illustrates an exemplary process for a dynamic distance manager, according to some examples;

FIG. 10B illustrates another exemplary process for a dynamic distance manager, according to some examples;

FIG. 10C illustrates another exemplary process for a dynamic distance manager, according to some examples;

FIG. 10D illustrates another exemplary process for a dynamic distance manager, according to some examples;

FIG. 10E illustrates another exemplary process for a dynamic distance manager, according to some examples;

FIG. 11 illustrates a block diagram for an exemplary wearable device with a dynamic distance manager, according to some examples; and

FIG. 12 illustrates an exemplary computer system suitable for use with a dynamic distance manager, according to some examples.

### DETAILED DESCRIPTION

Various embodiments or examples may be implemented in numerous ways, including as a system, a process, an apparatus, a user interface, or a series of program instructions on a computer readable medium such as a computer readable storage medium or a computer network where the program instructions are sent over optical, electronic, or wireless communication links. In general, operations of disclosed processes may be performed in an arbitrary order, unless otherwise provided in the claims.

A detailed description of one or more examples is provided below along with accompanying figures. The detailed description is provided in connection with such examples, but is not limited to any particular example. The scope is limited only by the claims and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

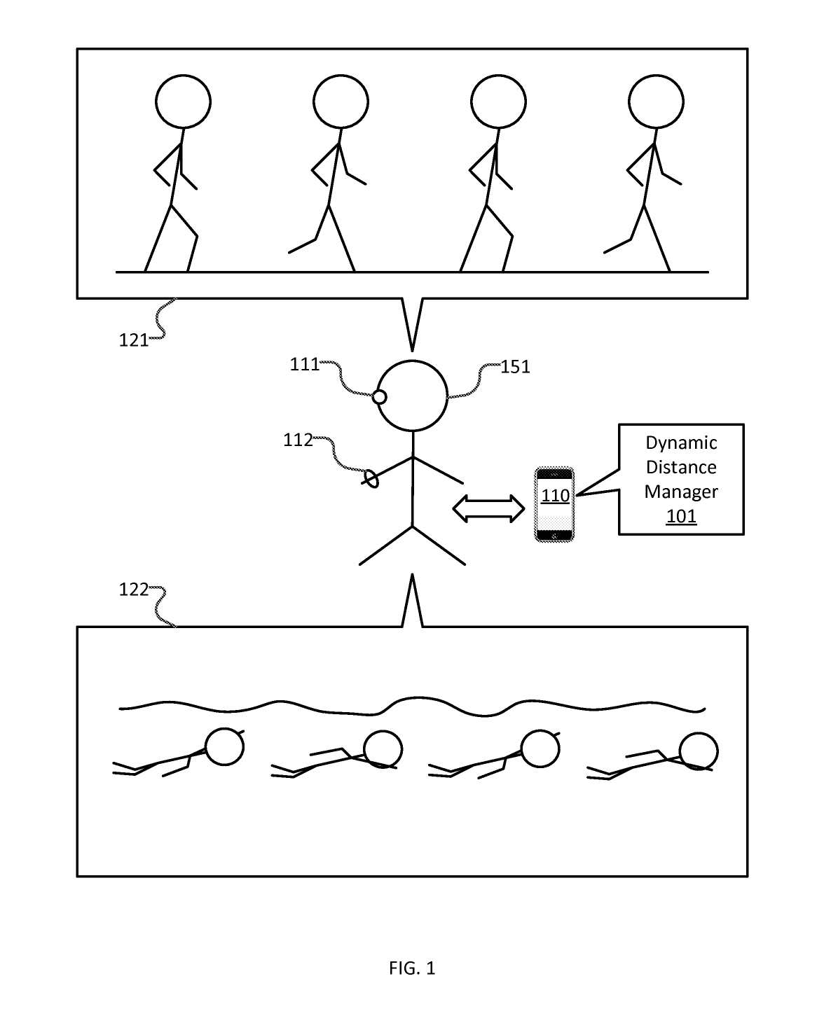

FIG. 1 illustrates an exemplary wearable device with a dynamic distance manager in some applications, according to some examples. As shown, FIG. 1 includes wearable devices 110-112, a dynamic distance manager 101, a user 151, and cyclical activities 121-122. Wearable devices 110-112 may be worn on or around an arm, leg, ear, or other bodily appendage or feature, or may be portable in a user's hand, pocket, bag or other carrying case. As an example, wearable device 110 is a smartphone, wearable device 111 is a headset, and wearable device 112 is a data-capable strapband. Other wearable devices such as a watch, data-capable eyewear, cell phone, tablet, laptop or other computing device may be used.

Wearable devices 110-112 may implement one or more facilities, sensing elements, or sensors, both active and passive, to capture various types of data from different sources. For example, data associated with physical motion or activity can be captured by an accelerometer, gyroscope, inertial sensor or other sensor. As another example, data associated with a physical location can be captured by a Global Positioning System receiver (GPS), or other location sensors for determining location within a cellular or micro-cellular network, which may or may not use GPS or other satellite constellations for fixing a position. Still, other sensors may be used and the above-listed sensors are not limiting. Sensors may be local or remote, or internal or external to wearable devices 110-112.

User 151 may perform various cyclical activities. A cyclical activity may be a series of repeated actions or motions, or an activity in which a pattern or set of substantially similar actions or motions occurs again and again. For example, cyclical activity 121 depicts walking, and cyclical activity 122 depicts swimming. Other examples of cyclical activities include running, swimming, ice-skating, bicycling and the like. Motion data associated with a cyclical activity may be captured by one or more sensors of wearable devices 110-112.

The motion data may include a number of motion units. A motion unit may be one cycle of the motion data, representing one set of similar actions that are repeatable, or repeatable displacements in a spatial coordinate system. For example, a motion unit may be a step of walking or running, that is, the motion of lifting of the left foot and the motion of putting down the left foot. Another motion unit may be the motion of lifting the right foot and the motion of putting down the right foot. As another example, a motion unit may be a stride of walking or running, that is, two steps, or the motion of lifting the left foot, the motion of putting down the left foot, the motion of lifting the right foot, and the motion of putting down the right foot. Each motion unit has a motion unit length. The motion unit length may be a distance or length of one motion unit. For example, a step may have a motion unit length of 0.7 meters, or a swim stroke may have a motion unit length of 1.2 meters.

Dynamic distance manager 101 may dynamically determine the motion unit length as a function of the number of motion units and the duration of making the motion units, updating the motion unit length at various time intervals during an activity. Since a time period may have a different number of motion units or a different duration from another time period, the motion unit length may vary over different time periods. Based on the motion unit lengths of individual time periods, dynamic distance manager 101 may determine the total distance of travel over all time periods. These time periods may also be called context windows, as discussed in detail below.

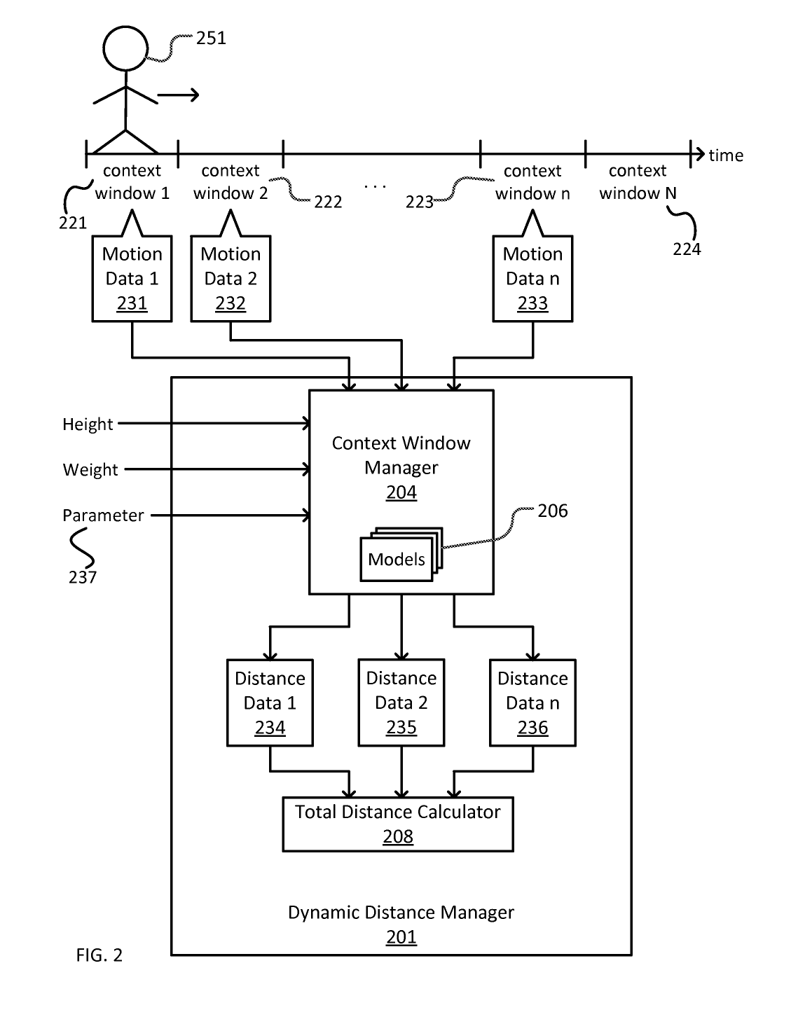

FIG. 2 illustrates an application architecture for an exemplary wearable device with a dynamic distance manager, according to some examples. As shown, FIG. 2 includes context windows 221-223, motion data 231-233, one or more parameters 237, a user 251, a context window manager 204, models 206, distance data 234-236, a total distance calculator 208, and a dynamic distance manager 201. Context window manager 204 and total distance calculator 208 may be implemented as part of dynamic distance manager 201 (as shown) or separate from dynamic distance manager 201. A context window may be a time period that is defined to have an approximate uniform motion unit length and may be used as the time interval for dynamic distance manager 201 to update its calculation of the motion unit length. In one embodiment, a context window is 3 seconds. In another embodiment, a context window is adjusted in such a way that the number of motion units encompassed in the context window is an integer.

In one embodiment, user 251 may be engaged in a cyclical activity, such as walking. Motion data may be captured for each context window. For example, motion data “1” 231 may be captured for context window “1” 221, motion data “2” 232 may be captured for context window “2” 222, and motion data “n” 233 may be captured for context window “n” 223. Motion data may be captured for each context window from the beginning of an activity to the end of the activity, and received by context window manager 204.

In addition, one or more parameters 237 may be received by context window manager 204. A parameter may be an attribute, characteristic or feature associated with user 251, including the way user 251 is moving or performing the activity. For example, a parameter may be the height, weight, or gender of user 251, the type of shoe user 251 is wearing while performing the activity (e.g., running shoes, hiking shoes, boots), or a physical disability of user 251 (e.g., on a crutch, on a walker, limping). As another example, a parameter may indicate whether the user is carrying the wearable device (e.g., in her hand, bag, etc.) or wearing the wearable device (e.g., on her arm, ear, waist, leg, etc.). Still other parameters may be used.

Context window manager 204 may use motion data 231-233 and parameter 237 to access models 206, which may be stored on a memory local to or integrated with context window manager 204 (as shown) or on a memory or database remote from context window manager 204. A model may be an representation, estimation or approximation of the relationship or association of the motion unit length with the number of the motion units and the duration of the motion units. In one embodiment, the number of motion units and the duration of the motion units may be used to calculate a cadence, and the model may be a representation of the association of the motion unit length with the cadence. A cadence may be the number of motion units per time unit, such as steps/second, stride/second, swim stroke/minute, or bicycle pedal/hour. Further, in other examples, different models may be used for persons with different parameters because persons with different parameters have different motion unit lengths. For example, a person who is taller may have a larger motion unit length for a given cadence due to longer legs. Thus for a cadence of, e.g., 1.8 steps/second, a person whose height is 1.5 m may have a motion unit length of 0.70 meters, and a person whose height is 1.8 m may have a motion unit length of 0.80 meters. As another example, a person who is wearing running shoes as opposed to dress shoes may have a larger motion unit length. As another example, a person using a walker versus a person with no physical disabilities may have a smaller motion unit length.

Context window manager 204 selects a model associated with parameter 237, and using the model determines the motion unit length for each context window 221-223. Because motion data 231-233 may be different for each context window 221-223, the motion unit length of each context window 221-223 may be different. For example, motion data “1” 231 of context window “1” 221 may indicate 5 steps and a duration of 3.04 seconds (cadence of 1.64 steps/second), motion data “2” 232 of context window “2” 222 may indicate 5 steps and a duration of 3.01 seconds (cadence of 1.66 steps/second), and motion data “3” 233 of context window “n” 223 may indicate 6 steps and a duration of 2.99 seconds (cadence of 2.00 steps/second). Context window manager 204 using the model 206 may determine that context window “1” 221 has a motion unit length of 0.70 meters based on a cadence of 1.64 steps/second, context window “2” 222 has a motion unit length of 0.75 based on a cadence of 1.66 steps/second, and context window “n” 223 has a motion unit length of 0.80 meters based on a cadence of 2.00 steps/second. Hence context window manager 204 dynamically determines the motion unit length associated with a context window.

Context window manager 204 may then determine distance data 234-236 of each context window 221-223 based on the motion unit length of context windows 221-223. Distance data 234-236 may indicate the distance of travel associated with context windows 221-223. Using the example above, the distance of travel of context window “1” 221 may be the motion unit length (0.70 meters) multiplied by the number of steps (5) of context window “1” 221, which is 3.5 meters. Similarly, the distance of travel of context window “2” 222 may be 5×0.75=3.75 meters, and the distance of travel of context window “n” 223 may be 6×0.80=4.8 meters.

Distance data 234-236 may then be received by total distance calculator 208, which adds or aggregates the distance of travel of each context window 221-223 to determine the total distance of travel. The total distance of travel may be the distance of travel over all context windows, such as the distance traveled from the beginning of an activity to the end of the activity, or the distance traveled from the beginning of when motion data is received to the end of when motion data is received. Using the example above, if there are three context windows 221-223, the total distance of travel is the sum of the distance of travel of context window “1” 221 (3.5 meters), the distance of travel of context window “2” 222 (3.75 meters), and the distance of travel of context window “n” 223 (4.8 meters), that is, 12.05 meters. In one example, the activity begins at context window “1” 221 and ends at context window “N” 224 (as shown), the distance of travel of each of the context windows from 221 to 224 may be determined using the process described above, and aggregated to determine the total distance of travel.

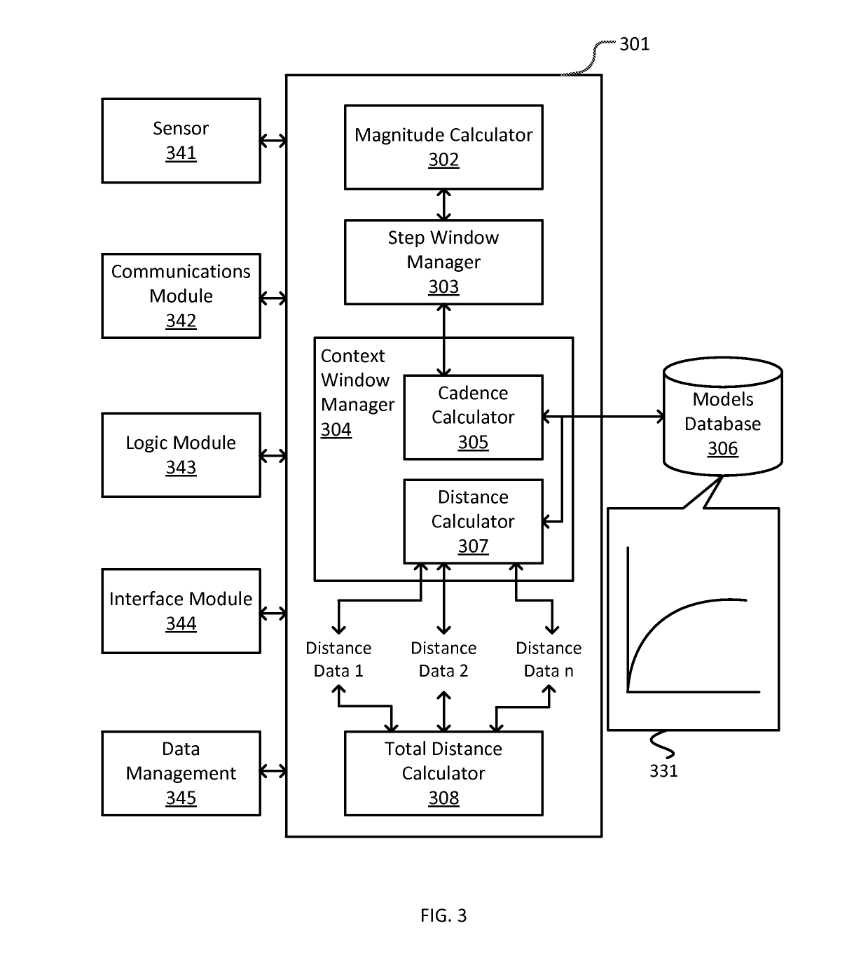

FIG. 3 illustrates another application architecture for an exemplary wearable device with a dynamic distance manager, according to some examples. As shown, FIG. 3 includes a dynamic distance manager 301, a magnitude calculator 302, a step window manager 303, a context window manager 304, a cadence calculator 305, a distance calculator 307, a total distance calculator 308, a models database 306, a model 331, a sensor 341, a communications module 342, a logic module 343, an interface module 344, and a data management module 345.

As described above, sensor 341 may be a motion sensor (e.g., accelerometer, gyroscope) or a location sensor (e.g., GPS). Sensor 341 may also be a sensor capable of detecting or capturing Bluetooth communications, Near Field Communications (NFC), temperature, audio, light, heart rate, altitude, or other sensory inputs. Sensor 341 may be a single or multiple sensors, and may include a variety of local or remote sensors. A local sensor may be a sensor that is fabricated, manufactured, installed, integrated or otherwise implemented with a wearable device (e.g., wearable devices 110-112 in FIG. 1). A remote sensor may be in data communication with the wearable device directly or indirectly (e.g., through a hub, network, etc.), such as, an accelerometer on another wearable device (e.g., an accelerometer on wearable device 112 may be remote from wearable device 110), a keyboard on a laptop or other distributed sensors. Interface module 344 may control or manage any user interface for transmitting or receiving data between the user and the wearable device. The user interface may be installed, integrated, fabricated or manufactured on the wearable device (e.g., a touchscreen on the wearable device), or may be installed on another wearable device, a laptop, another mobile device or other computing device that is in direct or indirect data communication with the wearable device (e.g., a mouse of a computer in data communication with the wearable device). The user interface may be implemented as a button, touchscreen, keyboard, sound, light or other device. Interface module 344 may also be used to detect or capture other sensory inputs. For example, a touchscreen may be used to detect the temperature of a user's finger. Communications module 342 may be used to transmit or receive data in a local or global network, using wired or wireless communications protocols (e.g., IEEE 802.11a/b/g/n (WiFi), WiMax, ANT™, ZigBee®, Bluetooth®, Near Field Communications (NFC), 3G, 4G, telecommunications, internet protocols, and others). Data management module 345 may be used to retrieve, receive or manage any memory or database, local or remote to the wearable device. Logic module 343 may be used to instruct, command, control, manage or provide other logic to dynamic distance manager 301, sensor 341, communications module 342, interface module 344, data management 345 and other components or devices. For example, logic module 343 may direct data from sensor 341 to magnitude calculator 302. Logic module 343 may also retrieve model 331 from model database 306.

Data from sensor 341, communications module 342, logic 343, interface module 344 and data management 345 may be received by dynamic distance manager 301. Motion data from sensor 341 may be received by magnitude calculator 302. For example, sensor 341 may detect a motion vector with more than one component or axes, such as a 2- or 3-axis accelerometer. Magnitude calculator 302 may determine a magnitude of the motion vector. For example, a 3-axis accelerometer may give a motion vector as an output, such as a reading of the acceleration for each of three axes, x, y, and z, and magnitude calculator 302 may determine the magnitude using a formula, e.g., \sqrt{x²+y²+z²}. Magnitude calculator 302 may also determine a magnitude of the motion vector that takes into account the orientation of the sensor 341. Magnitude calculator 302 may determine the direction pointing down by looking for the component or axis with the greatest gravitational influence, and then calculate a weighted average of the components of the motion vector, weighted by the direction that is pointing down.

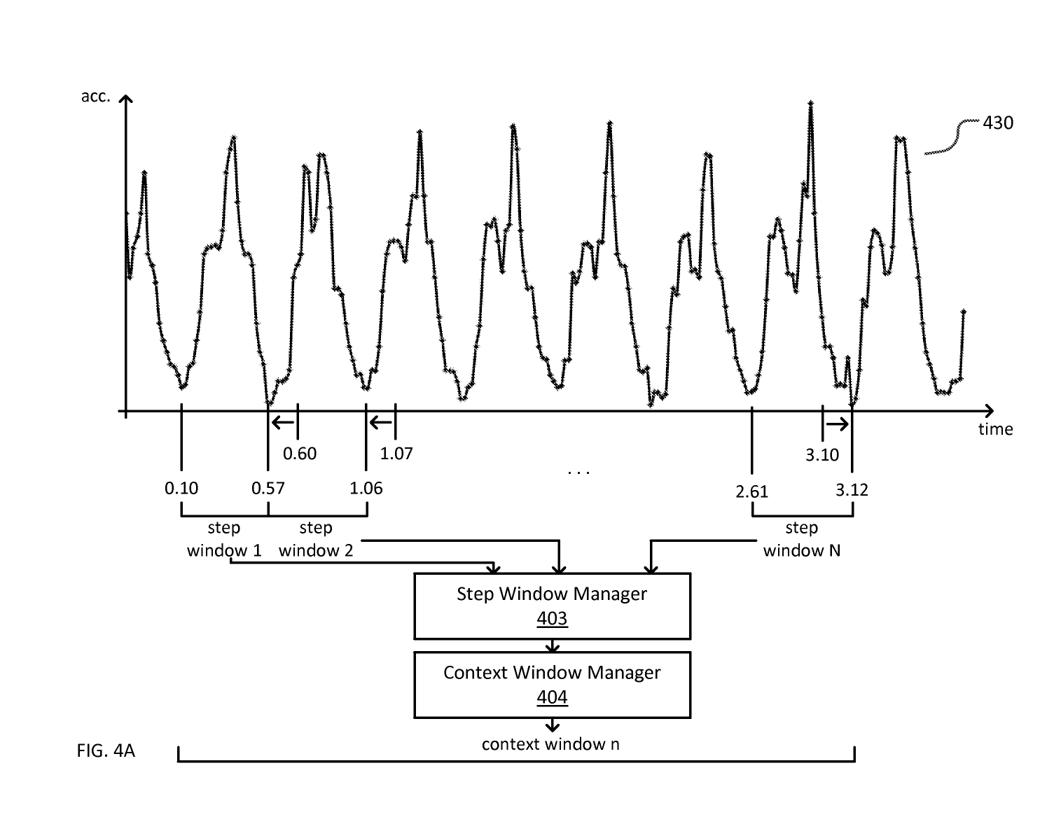

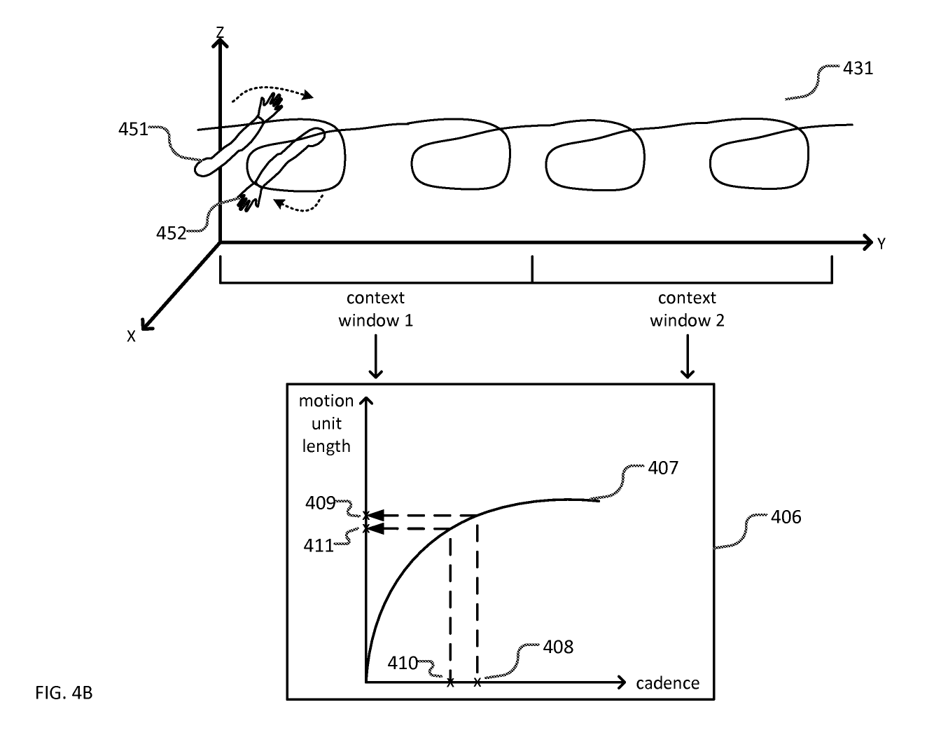

Motion data from sensor 341, or the magnitude of the motion vector from magnitude calculator 302, may be received by step window manager 303. Step window manager 303 may determine the number of motion units within a step window. A step window may be a fraction or portion of a context window. In one embodiment, a step window may be 0.5 seconds and a context window may be 3 seconds. Step window manager 303 may determine the number of motion units by counting the number of cycles made by the magnitude of the motion vector (see FIGS. 4A & 4B). Step window manager 303 may also determine the number of motion units by counting the number of cycles made by one component of the motion vector, or each component of the motion vector. Step window manager 303 may also determine the duration of each step window.

Other types of data may be used in conjunction with or in lieu of motion data to determine the number of motion units of a step window. For example, a user's foot hitting the ground may make a thump detected by an audio sensor, which may be used to determine or confirm impact associated with a step. As another example, the heart rate may increase each time a swimmer raises his hand above the water to make a swim stroke. Step window manager 303 may use other types of data to determine or verify the number of motion units of each step window. For example, step window manager 303 may count one motion unit when there is one cycle in the motion data and one thump sound.

The number of motion units of each step window and the duration of each step window are received by context window manager 304. A plurality of step windows may form a context window. Context window manager 304 may add or aggregate the number of motion units of each step window to determine the number of motion units of the context window. Context window manager 304 may add or aggregate the duration of each step window to determine the duration of the context window. In another embodiment, context window manager 304 may determine the number of motion units of a context window and the duration of the context window directly from the motion data or the magnitude of the motion vector, without step window manager 303. For example, context window manager 304 may count the number of cycles made by the motion data over the context window. Context window manager 304 may also determine the duration of the context window. Then cadence calculator 305 may determine the cadence of the context window by dividing the number of motion units of the context window by the duration of the context window.

Model database 306 may include one or more models 331 to be used for determining motion unit length. A model may represent an association of the motion unit length with the number of motion units and the duration of the motion units. In one embodiment, a model may represent an association of the motion unit length with the cadence (motion units per time). In another embodiment, a model may represent an association of the motion unit length with duration per motion unit, or another number or representation related to the number of motion units and the duration of the motion units. A model may also be associated with one or more parameters describing, related to or associated with the user. Parameters associated with the user may be received from sensor 341, communications module 342, logic module 343, interface module 344, or data management 345. For example, the user may input into interface module 344 using a keyboard of a computer in data communication with the wearable device that he is 5′ tall and 130 lbs in weight. For example, Bluetooth or NFC data may be used to detect a type of shoe being worn if the shoe has an identifier or label that is being transmitted using Bluetooth or NFC. The identifier may also include the brand of the shoe or the model number of the shoe. For example, data management 345 may access a memory storing a user profile, which includes the user's gender and other personal information. Context window manager 304 may access model database 306 to identify a model associated with the parameters of the user, and based on this model use the cadence of the context window to determine the motion unit length of the context window.

The motion unit length of the context window may be received by distance calculator 370. Distance calculator 307 determines the distance of travel of the context window. Distance calculator 307 may multiply the motion unit length by the number of motion units of the context window. Cadence calculator 305 and distance calculator 307 may be implemented or installed as part of context window manager 304 (as shown) or may be separate from context window manager 304. Model database 306 may be stored remotely (e.g., on a server) and may be accessed by context window manager 304 using wired or wireless data communications. Model database 306 may also be local to context window manager 304 or dynamic distance manager 301.

The distance of travel of each context window may be received by total distance calculator 308. “Distance data 1” may represent the distance of travel of context window “1”, “distance data 2” may represent the distance of travel of context window “2”, and “distance data n” may represent the distance of travel of context window “n”. Total distance calculator 308 adds or aggregates the distance of travel of each context window to determine the total distance of travel.

Interface module 344 may display or present information relating to the total distance of travel, distance of travel of each context window, number of motion units of each context window, or any of the data described above. For example, a screen may display that 100 meters has been walked. As another example, a light may flash orange to indicate that the user has walked a farther distance today than she did yesterday. As another example, a speaker may produce a sound recording motivating the user to walk more steps if the number of steps is less than his average number of steps, or may produce a music or song with a faster beat.

FIG. 4A illustrates exemplary motion data for an activity for use by an exemplary wearable device with a dynamic distance manager, according to some examples. FIG. 4A includes motion data 430, step window manager 403 and context window manager 404. Motion data 430 may be the magnitude of a motion vector, determined by magnitude calculator 302 (FIG. 3). Motion data 430 may also be one component of a motion vector, such as the component of the motion vector that is pointing down. Motion data 430 may also be data directly received from a sensor such as a 1-axis accelerometer. In one embodiment, motion data 430 indicates data representing acceleration as a function of time for walking. For example, a person walking may raise one foot, and step or fall onto it, and then raise another foot, and step or fall onto it. An accelerometer may detect the rising and falling as an increase and decrease in acceleration, thus forming cycles in the motion data 430. In one embodiment, the user may be carrying rather than wearing the wearable device. In this case, a sensor of the wearable device may be loosely associated with the user's steps. When a user raises one foot, the sensor may not detect the effect until after a delay or the effect may be dampened. The increase and decrease in acceleration detected by the accelerometer and thus the cycles in the motion data may be less marked. One cycle of the motion data may indicate one motion unit or step. A motion unit may be between two minimums in the acceleration, two maximums in the acceleration, or any other two data points that are repeating or cyclical in the acceleration. For example, as shown, one cycle extends from a minimum at 0.10 seconds to another minimum at 0.57 seconds.

In one embodiment, the duration of each step window is adjusted in such a way that the number of motion units in each step window is an integer. For example, as shown in FIG. 4A, a nominal step window is set to be 0.5 seconds. “Step window 1” may begin at 0.10 seconds. Step window manager 303 may count the number of motion units made until it reaches 0.5 seconds. Then it may identify the closest transition of a motion unit (i.e., the end of one motion unit and the beginning of the next motion unit), and may either shorten or lengthen the step window so that the step window ends at the transition of the motion unit. As shown, “step window 1” nominally ends at 0.6 seconds. The closest transition of a motion unit is at 0.57 seconds. Therefore step window manager 403 may shorten “step window 1” to end at 0.57 seconds. Hence “step window 1” may contain an integer number of motion units, in this example, being one.

“Step window 2” may then begin immediately after “step window 1”. Using this example, it begins at 0.57 seconds. “Step window 2” may nominally end at 0.57+0.5=1.07 seconds. Step window manager 403 may count the number of steps until it passes 1.07 seconds. Step window manager 403 may then identify the transition of a motion unit closest to 1.07 seconds, in this example, 1.06 seconds. Step window manager 403 may adjust “step window 2” to end at 1.06 seconds. Step window manager 403 may continue this process for each step window until it reaches the end of a context window. Still other implementations may be possible. For example, the beginning of “step window 2” may be a few milliseconds after the end of “step window 1”.

The number of motion units and duration of each step window may be received by context window manager 404. In one embodiment, the duration of each context window is adjusted in such a way that the number of step windows in each context window is an integer. For example, as shown in FIG. 4A, a nominal context window is 3 seconds. “Context window n” starts at 0.10 seconds and nominally ends at 3.10 seconds. When the time passes 3.10 seconds, context window manager 404 determines the closest transition of a step window, and adjusts the length of the context window so that the context window ends at the transition of the step window. For example, the closest transition of a step window is at the end of “step window N,” at 3.12 seconds. Context window manager 404 may lengthen “context window n” to end at 3.12 seconds. Hence “context window n” may contain an integer number of step windows. The next context window (e.g., “context window n+1”) may begin immediately afterwards, that is, at 3.12 seconds. In another embodiment, “context window n+1” may begin a time period after the end of “context window n”. In another embodiment, context windows may be used without using step windows. For example, the duration of each context window is adjusted in such a way that the number of motion units in each context window is an integer, using processes similar to above. Still other processes or implementations may be possible.

FIG. 4B illustrates exemplary motion data for another activity for use by an exemplary wearable device with a dynamic distance manager, according to some examples. FIG. 4B includes motion data 431, user's arm 451-452, model 406, relationship 407, cadence data points 408 and 410, and motion unit length data points 409 and 411. In one embodiment, motion data 431 for a person swimming is captured over two context windows by a sensor. User's arm 451 may first be raised, and user's arm 452 may then swing to the back, forming a swim stroke. A cadence for each of “context window 1” and “context window 2” may be determined using the processes described above. For example, the cadence for “context window 1” may be data point 410, and the cadence for “context window 2” may be data point 408. Relationship 407 may represent an association of motion unit length with cadence in model 406. Data points 410 and 408 are traced up to relationship 407, and then across to data points 411 and 409. Data point 411 may be the motion unit length of “context window 1,” and data point 409 may be the motion unit length of “context window 2.” Hence, the motion unit length of each context window may be determined.

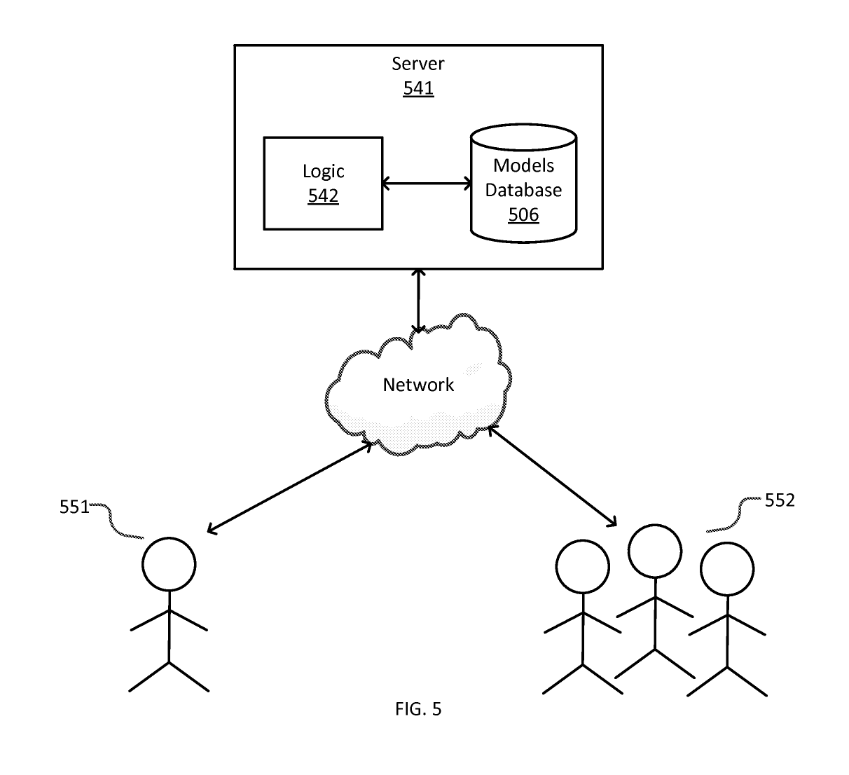

FIG. 5 illustrates a network for use by a plurality of exemplary wearable devices with a dynamic distance manager, according to some examples. As shown, FIG. 5 includes server 541, logic 542, model database 506, user 551, and other users 552. As described above, model database 506 may be remote from the wearable device of user 551. In one embodiment, model database 506 is saved on server 541. Server 541 may be accessible by the wearable devices of user 551 and other users 552 through a network. The network may be wired or wireless, local or global, private or public. It may use data communication protocols such as IEEE 802.11a/b/g/n (WiFi), WiMax, ANT™, ZigBee®, Bluetooth®, Near Field Communications (NFC), 3G, 4G, telecommunications, internet protocols, and others. Logic 542 may access model database 506 to retrieve data requested by the wearable devices of user 551 and 552. Hence, models may be shared with a group of users. Once downloaded from server 541, the model may be stored in a local memory of the wearable device of a user. Thus, the model may subsequently be locally accessed by the wearable device.

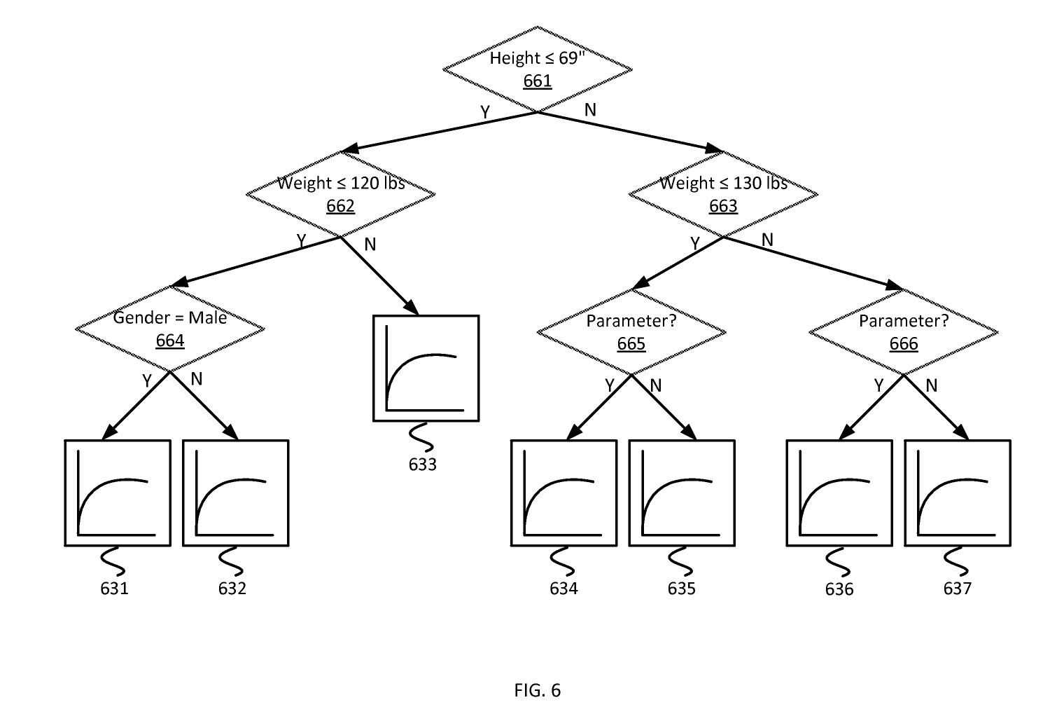

FIG. 6 illustrates an exemplary decision tree for use by an exemplary wearable device with a dynamic distance manager, according to some examples. As shown, FIG. 6 includes nodes 661-666, and models 631-637. A decision tree may be used to determine a model associated with the parameters of the user from a plurality of models to be used for determining the motion unit length of the user. Parameters associated with the user may be input to the decision tree. At 661, the decision tree may determine whether the height is less than or equal to 69″. If yes, the process goes to 662, and the decision tree may determine whether the weight is less than or equal to 120 lbs. If yes, the process goes to 664, and the decision tree may determine whether the gender is male. If yes, then model 631 may be used; if no, then model 632 may be used. A decision tree can be made up of a variety of levels. For example, going back to 662, if the answer is no, the decision tree need not have another level, and it may indicate that model 633 is to be used. The questions of the decision tree need not be symmetrical. For example, going back to 661, if the answer is no, the decision tree need not make the same determination as 662; it may determine if the weight is less than or equal to 130 lbs. As another example, going to 665 and 666, each may determine a different parameter. For example, 665 may determine a type of shoe, and 666 may determine a physical disability. The decision tree will then identify model 634, 645, 646 or 637 to be used.

In one embodiment, a table may be used to determine a model associated with the parameters of the user. Parameters associated with the user may be input into the table. The table may look up a table entry with matching parameters, or a table entry that best fits the parameters. The table entry may then indicate which model to use. For example a table entry may have the following parameters: Weight is over 120 lbs, gender is female, height is less than 69″. This table entry may indicate a certain model from a plurality of models. The parameters of a user may be as follows: weight is 125 lbs, gender is female, and height is 68″, and the parameters may be input into the table. A look up may be performed and may determine that the table entry described above matches the parameters of the user. Hence, the model indicated by the table entry may be used.

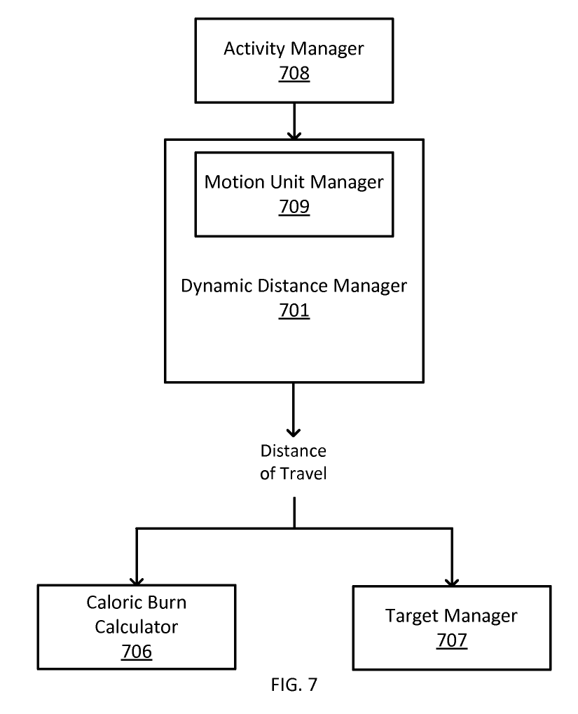

FIG. 7 illustrates exemplary applications of a dynamic distance manager, according to some examples. As shown, FIG. 7 includes dynamic distance manager 701, activity manager 708, caloric burn calculator 706, and target manager 707. Activity manager 708 may determine the activity being performed by the user from motion data and other data received from one or more sensors coupled to the wearable device of the user. In one embodiment, a user may input through a user interface the type of activity she is engaged in. For example, prior to beginning a run, the user uses the touchscreen of a smartphone to input that she is beginning a run, and at the end of a run, the user inputs that she is ending the run.

In another embodiment, activity manager 708 may have a pattern library storing one or more patterns representing one or more activities. A pattern may be a set of one or more attributes having a set value or range of values. For example, a pattern representing walking may include motion data similar to data 331 (FIG. 4A). As another example, a pattern representing walking may include motion data similar to data 331, a maximum number of cycles of the motion data being 7 cycles per 3 seconds, and a thumping sound which has a frequency matching the frequency of the cycles of the motion data. As another example, a pattern representing running may be similar to a pattern representing walking, but with a maximum number of cycles of the motion data being 3 cycles per 0.5 seconds. As another example, a pattern representing hiking may be similar to a pattern representing walking, but with location data or map data indicating that the user is at a park. In one embodiment, a pattern may also represent a parameter or characteristic of the user, including the way the user is performing the activity. For example, a pattern representing a person walking with an injured left ankle may include motion data that is similar to one normal step shown in 331 (FIG. 4A) followed by an irregular step, then a normal step, then an irregular step. Still other patterns may be used.

Motion data and other data may be received from the sensors by activity manager 708, and activity manager 708 may compare the data with the patterns in the pattern library and determine whether a pattern matches the motion data, or which pattern best fits the motion data. Based on the match, activity manager 708 determines the activity being performed by the user. Activity manager 708 may also a parameter or characteristic of the user performing the activity. Examples for determining activities are disclosed, for example, in U.S. patent application Ser. No. 14/064,189 entitled “Data-Capable Band Management in an Integrated Application and Network Communication Data Environment” filed Oct. 27, 2013.

Dynamic distance manager 701 may have a motion unit manager 709. Data representing the activity may be received by motion unit manager 709. Motion unit manager 709 may determine what attribute to look for in the motion data to identify a motion unit based on the activity. For example, for walking, motion unit manager 709 may determine that a minimum in acceleration indicates the transition of a motion unit. For swimming, motion unit manager 709 may determine motion data indicating the raising of an arm 451 (FIG. 4B) indicates the transition of a motion unit. Dynamic distance manager 701 may then determine the total distance of travel using the processes described above. Dynamic distance manager 701 may determine the motion unit length of each context window, then the distance of travel of each context window based on the motion unit length, and then the total distance of travel.

A distance of travel of a context window or the total distance of travel may be received by caloric burn calculator 706 or target manager 707. Caloric burn calculator 706 may determine the number of calories burned as a function of the distance of travel.

Caloric burn calculator 706 may access a memory storing a METS (metabolic equivalent of task) table, indicating METs values for different activities performed at different intensities. For example, the METs table may indicate that walking at 2.7 kilometers per hour (km/h) is 2.3 METS, walking at 4.8 km/h is 3.3 METS, and running is 8.0 METS. From the METS value, the number of calories burned may be calculated using a formula, e.g., Calories=(Weight of person)×(Duration of the activity)×METS. The METS table may also take into account other parameters associated with the user (e.g., height, physical health, resting metabolic rate, etc.). For example, caloric burn calculator 706 receives data indicating that 1 km was traveled over 15 minutes (0.25 hours) by a user walking, and the user weighs 130 lbs (about 59 kg). Caloric burn calculator 706 may determine that the speed was 1/0.25=4 km/h. Caloric burn calculator 706 may look up a METS table for the METS value of walking at 4 km/h and determine that it is 3 METS. Caloric burn calculator 706 may determine that the caloric burn is 59×0.25×3=44.35 kcal. In another embodiment, caloric burn calculator 706 may determine the caloric burn for each context window and aggregate this to determine the total caloric burn. Caloric burn calculator 706 may receive data representing the distance of travel of a first context window, calculate the speed, and look up the METS value for performing the activity at that speed. Caloric burn calculator 706 may then calculate the caloric burn for the first context window. Caloric burn calculator 706 may then receive data representing the distance of travel of a second context window, and similarly calculate the caloric burn for the second context window. Caloric burn calculator 706 may determine the sum of all caloric burns of each context window to determine the total caloric burn. Other methods for determining the number of calories burned from the distance of travel may also be used.

Target manager 707 may determine whether the user has achieved a certain target. The target may be a distance of travel for a certain activity. The target may be set by the user or an application (such as a fitness application available on a marketplace). The target may also be set in a “competition” with other users. Wearable devices of other users may be in data communication with the wearable device of the user through a network, such as the network shown in FIG. 5. A wearable device may communicate through the network that a first user would like to start a competition, for example, to see who can walk 1000 meters first. Another wearable device may respond through the network that a second user would like to join the competition. Hence the target of the second user may be set by the first user. Target manager 707 may compare the distance of travel with the target to determine whether the target has been achieved. In another embodiment, the target may be a number of calories burned. The number of calories burned or whether the target has been achieved may be displayed on a user interface coupled to the wearable device of the user. For example, a message on a screen of the wearable device may display the number of calories burned. As another example, a motor of the wearable device may provide a vibration when the target is achieved. Other applications of the dynamic distance manager 701 may also be used.

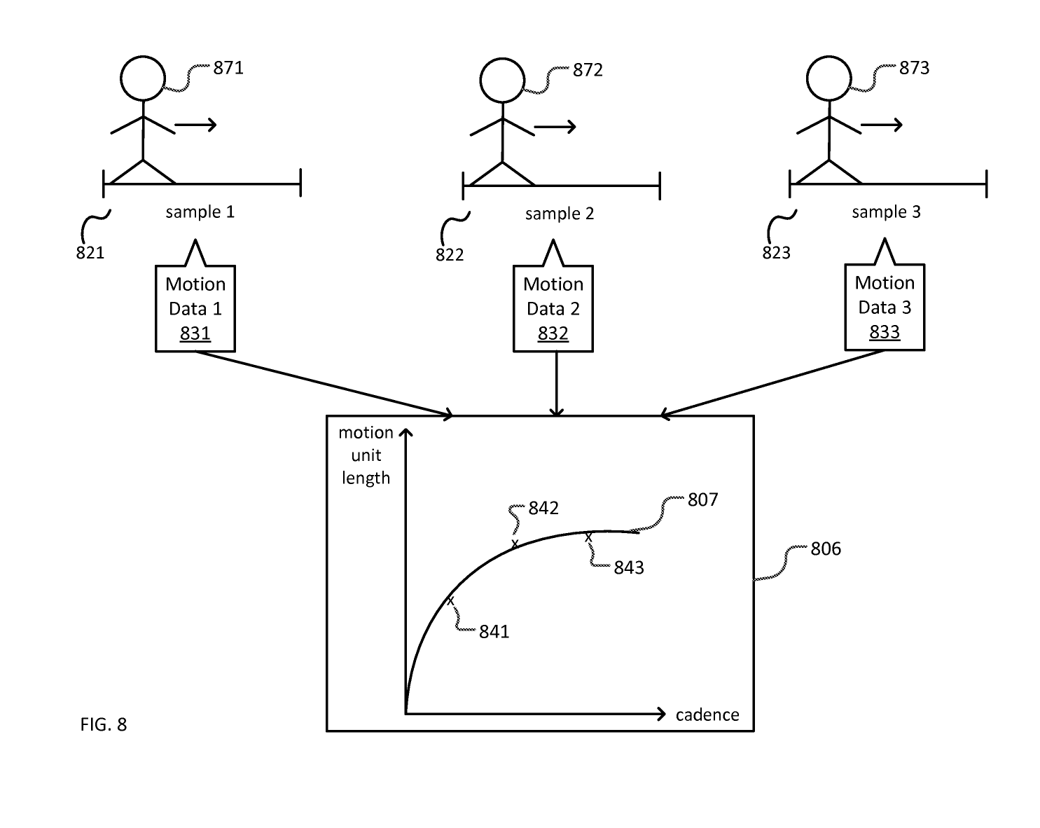

FIG. 8 illustrates exemplary motion data for use in creating an exemplary model for use by a dynamic distance manager, according to some examples. As shown, FIG. 8 includes persons 871-873, samples 821-823, motion data 831-833, model 806, relationship 807, and data points 841-843. A model may be created by fitting a curve or other relationship from a plurality of sample data points. The relationship may be based on linear regression, non-linear regression, ordinary least squares (OLS), iterative approaches, interpolation, smoothing, statistical models or other methods. Sample data points may be captured by detecting motion data for performing an activity for a given distance by persons 871-873. For example, person 871 may walk a given distance, and motion data “1” 831 is captured. Similarly, persons 872 and 873 may walk a given distance, and motion data “2” 832 and motion data “3” 833 may be captured. Persons 871-873 may be the same or different persons, and may be walking at the same or different speeds. The given distance for samples 821-823 may all be the same or may be different. Motion data 831-833 may include the number of steps taken, the duration of taking those steps, and the given distance.

In one embodiment, the number of steps taken and the given distance (adjusted or non-adjusted) may be determined by one or more sensors worn or carried by persons 871-873. For example, motion data may be captured by an accelerometer, and the number of cycles of the motion data indicates the number of steps taken, using a process similar to the process described above with respect to FIGS. 4A & 4B. Location data may be captured by a GPS or other location sensor, indicating the distance traveled. For example, a GPS may detect that a person has walked 100 meters. In another embodiment, persons 871-873 walk a given distance on a treadmill. A screen of the treadmill showing the distance traveled on the treadmill may be used to determine when the given distance has been traveled.

In still another embodiment, the number of steps taken and the given distance may be determined by a person reviewing samples 821-823. For example, the person reviewing samples 821-823 may count the number of steps taken. The counting may also be done using a video recording of samples 821-823. Also a person reviewing samples 821-823 may ask persons 871-873 to walk on a street from Landmark A (e.g., fire hydrant) to Landmark B (e.g. mailbox). Landmarks A and B may indicate the beginning and end points of the given distance used for samples 821-823. The distance from Landmark A to Landmark B may be measured by a distance measuring tool, such as a wheel, electronic tool, laser or other device. In one embodiment, the given distance may be adjusted in such a way that the number of steps taken is an integer. For example, person 871 may start walking before Landmark A, pass Landmark A, pass Landmark B, and end walking after Landmark B. The given distance may be adjusted to start at the transition of a step closest to Landmark A and to end at the transition of a step closest to Landmark B. To determine the distance from the closest transition of a step to the respective Landmarks, rulers may be placed near Landmarks A and B, viewable by a person observing persons 871-873 walking. For example, a person reviewing sample “1” 821 (whether live or on video) may determine that a foot of person 871 hit the ground 5 cm before Landmark A and then the other foot hit the ground 32 cm after Landmark A, using the ruler. Also the person reviewing sample “1” 821 may determine that a foot of person 871 hit the ground 25 cm before Landmark B and 14 cm after Landmark B. Then the transition of a step closest to Landmark A is 5 cm before Landmark A, and the transition of a step closest to Landmark B is 14 cm after Landmark B. The distance between Landmarks A and B may be measured to be, e.g., 900 m. Then the given distance used for sample “1” 821 may be the distance between Landmarks A and B plus the distance between the closest transition of a step and Landmark A plus the distance between the closest transition of a step and Landmark B, e.g., 900 m+5 cm+14 cm=900.19 m. Hence, the given distance of 900.19 m encompasses an integer number of steps.

In one embodiment, a cadence may be calculated for each motion data 831-833 by dividing the number of steps by the duration of each respective sample. A motion unit length may also be calculated for each motion data 831-833 by dividing the given distance by the number of steps of each respective sample. For example, for sample “1” 821, the cadence is the number of steps taken by person 871 divided by the duration that person 871 took to make those steps, and the motion unit length is the given distance traveled by person 871 divided by the number of steps taken by person 871. The cadence and motion unit length of each sample may be plotted in a graph or model 806 showing motion unit length (on the y-axis) as a function of cadence (on the x-axis). Sample “1” 821 may correspond to data point 841, sample “2” 822 may correspond to data point 842, and sample “3” 823 may correspond to data point 843. A relationship 807 may be fitted to the data points 841-843. Hence model 806 may be used by dynamic distance manager to determine motion unit length as a function of cadence. As described above, a model may be an association of the motion unit length with the number of motion units taken and the duration of taking the motion units. For example, a model may be an association of the motion unit length with the duration or length of time per motion unit (e.g., seconds/step).

In one embodiment, one or more parameters may be commonly associated with persons 871-873. For example, persons 871-873 are all females who are shorter than 69″ and weigh less than 120 lbs. Model 806 may be associated with these one or more parameters, and added to a decision tree (FIG. 6). For example, model 806 may be placed at the position of model 632 in FIG. 6, that is, where the decision tree indicates less than 69″ at 661, less than 120 lbs at 662, and gender not equal to male at 664. In one embodiment, model 806 may create a new node on the decision tree. For example, persons 871-873 are all females who are shorter than 69″, weigh less than 120 lbs, and wearing running shoes. Then at 664, after the arrow pointing to N (indicating gender is not male), an additional node determining “Are running shoes being worn?” may be added. From this new node, an arrow pointing to Y (indicating running shoes are being worn) may lead to the new model 806. In another embodiment, model 806 may be added to a table associating models with parameters. Still other methods for creating a model associated with one or more parameters may be used.

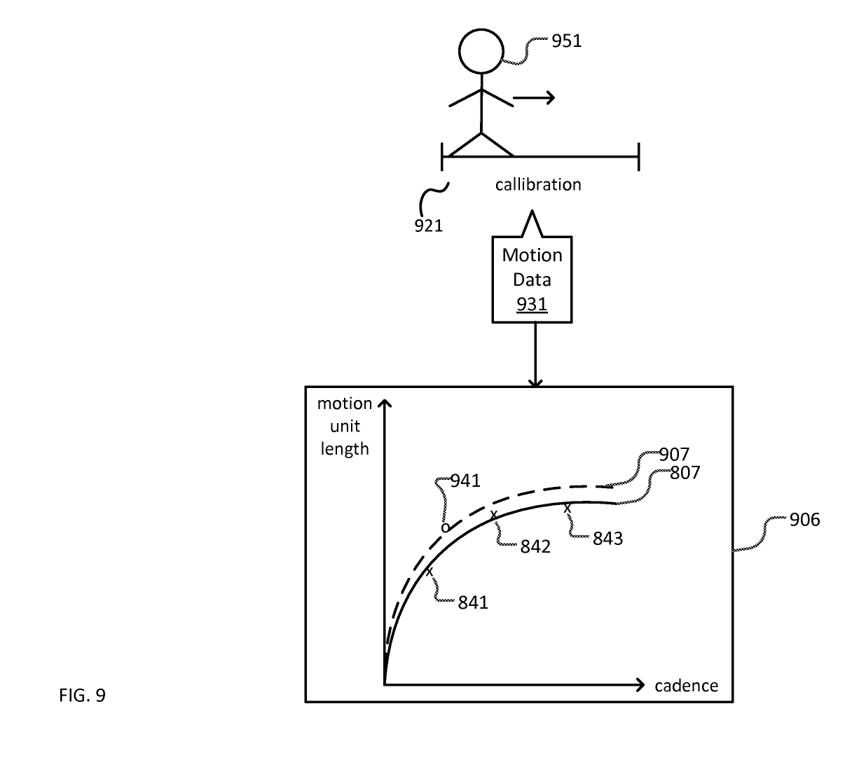

FIG. 9 illustrates exemplary motion data for use in adjusting or calibrating an exemplary model for use by a dynamic distance manager, according to some examples. As shown, FIG. 9 includes a person or user 951, calibration 921, motion data 931, model 906, relationships 807 and 907, data points 841-843 and 941. A model may be adjusted or calibrated by adding data points to the model and re-fitting a curve or other relationship to the new data points. The relationship of the adjusted model may be based on the same or a different method (e.g., linear regression, non-linear regression, OLS, etc.) than the relationship of the original model. Model 906 may be a model associated with one or more parameters of user 951, to be used to determine the motion unit length of user 951. Before being adjusted, model 906 have data points 841-843 and relationship 807 (such as in FIG. 8). User 951 may have a unique gait, attribute or other parameter, such that his motion unit length as a function of cadence is different from persons 871-873 (FIG. 8). User 951 may input through a user interface of the wearable device that he would like to enter a calibration mode. Model 906 may then be adjusted or calibrated to fit or take into account user 951's characteristics.

For example, user 951 may walk a given distance, and motion data 931 is captured. Motion data 931 may be captured by a wearable device worn or carried by user 951 and may include the number of motion units taken and the duration of the motion units. A cadence may be calculated by dividing the number of motion units by the duration. A motion unit length may be calculated by dividing the given distance by the number of motion units. The cadence and the motion unit length may be plotted in model 906, making, for example, data point 941. A new relationship 907 may be fitted to data points 841-843 and 941. Hence model 906 takes into account user 951's unique characteristics, and model 906 may be subsequently used by the dynamic distance manager of the wearable device of user 951 to determine the total distance of travel. In one embodiment, calibration may be repeated for different speeds of travel. For example, user 951 may walk a given distance using a normal speed, and motion data 931 is captured. Motion data 931 is plotted as data point 941, as described above. User 951 may then walk a given distance using a faster speed, and another motion data is captured and is plotted as another data point in model 906. A new relationship may be fitted to the data, including data point 941 and the other data point corresponding to the motion data associated with the faster speed. In another embodiment, old data points that are found to be inaccurate may be removed, and a new relationship may be fitted to the remaining data points.

In one embodiment, model 906 may further be shared with other users. For example, model 906 may be transmitted to server 541 (FIG. 5). Other users 552 may then access model 906 over the network. In another embodiment, attributes or parameters of user 951 may be input to the wearable device before or during calibration, and then associated with model 906. Model 906 may then be added to a decision tree with the associated parameters, as described above. Other users 552 having these parameters in common may then access model 906 on the server.

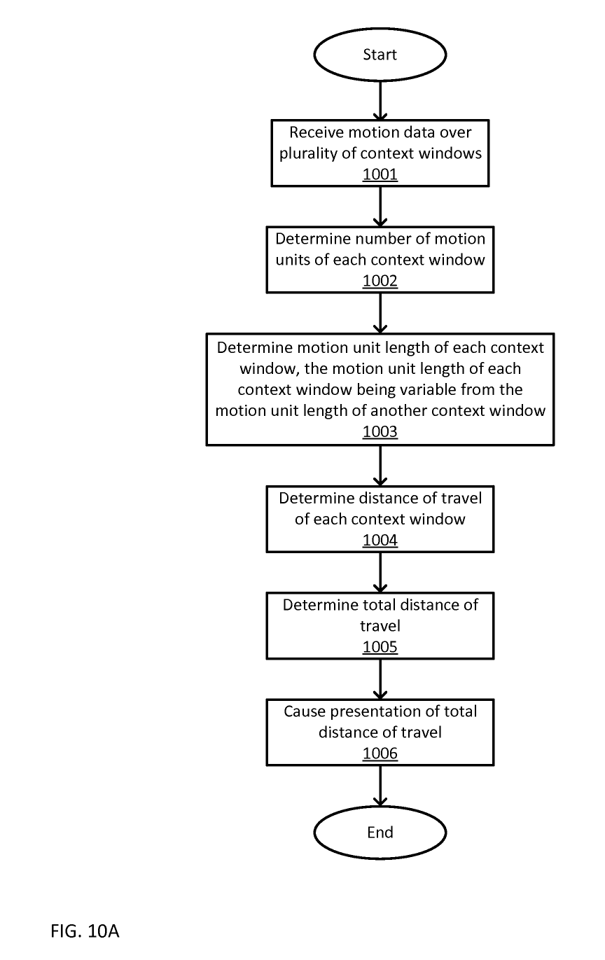

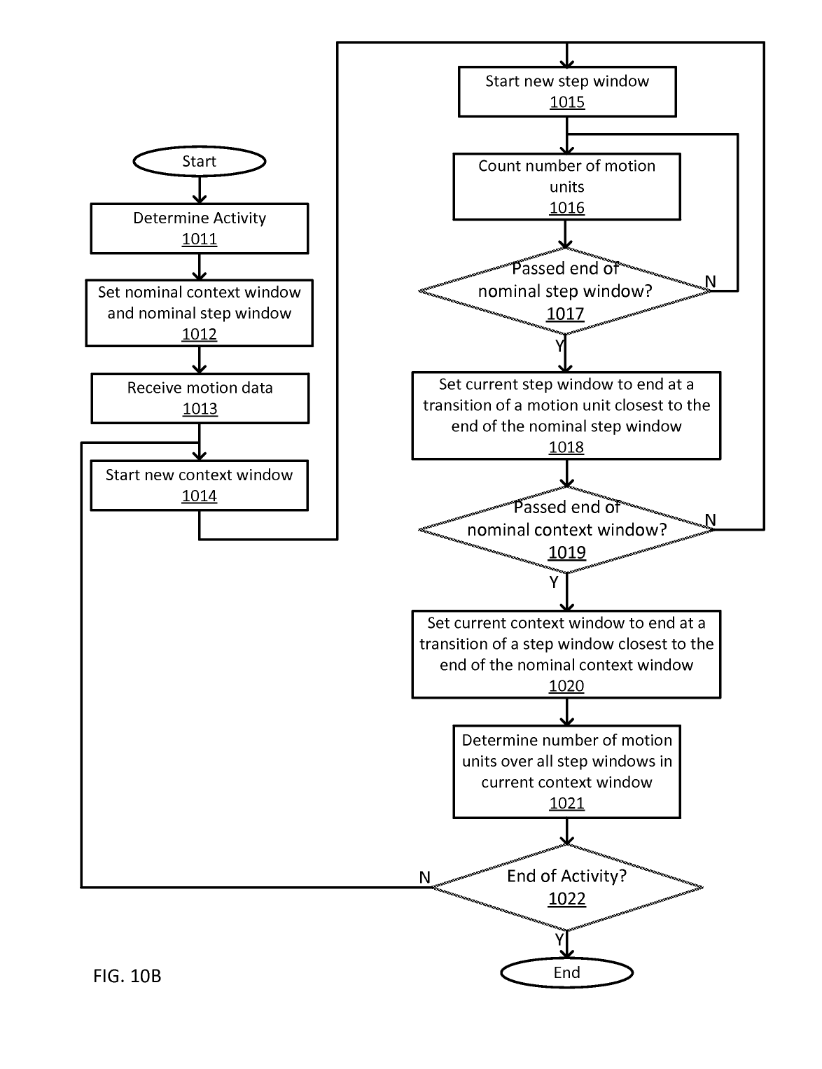

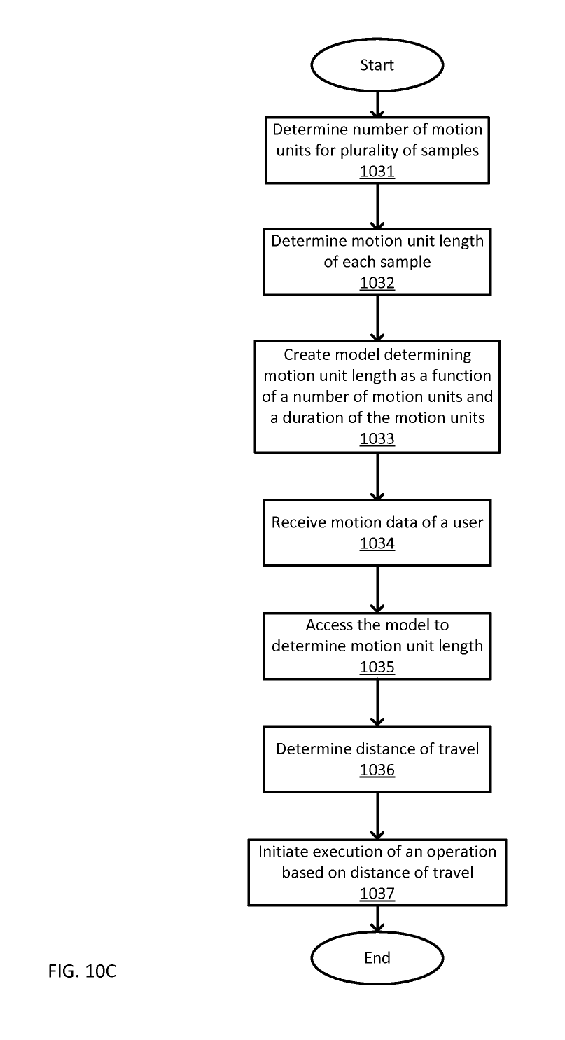

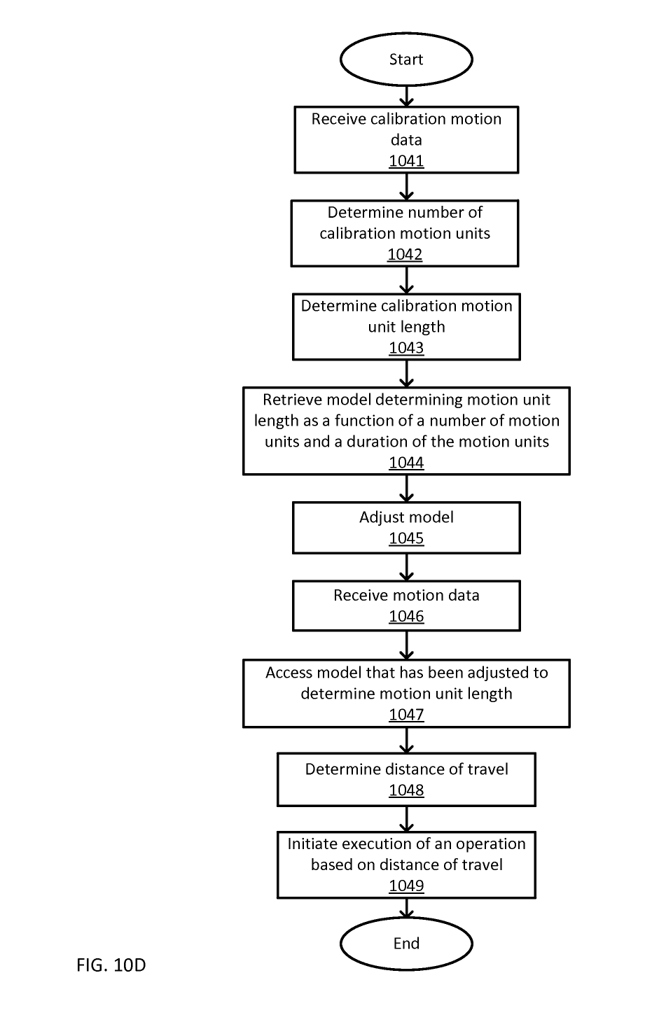

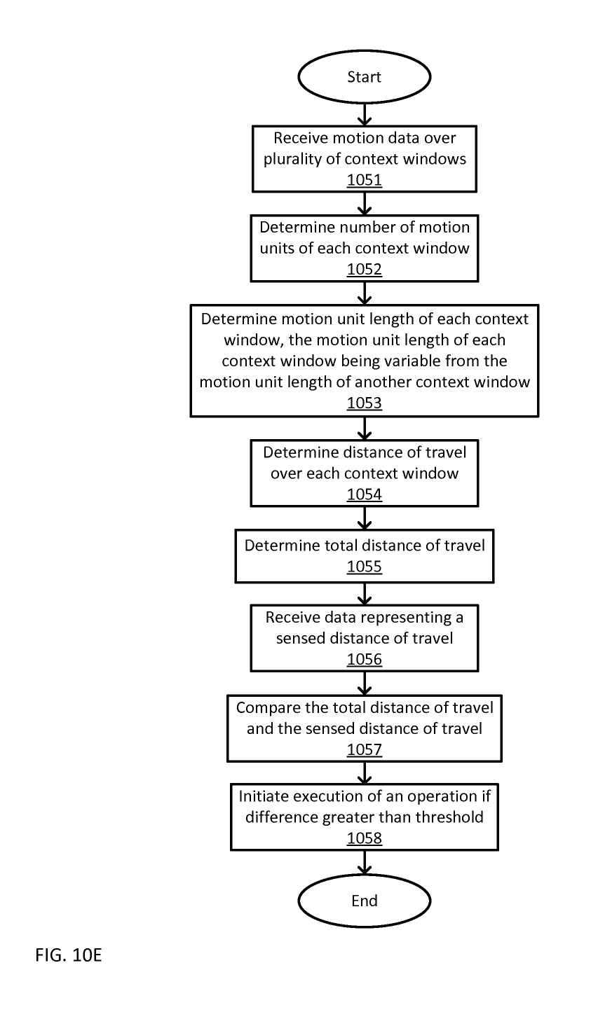

FIG. 10A illustrates an exemplary process for a dynamic distance manager, according to some examples. At 1001, motion data is received over a plurality of context windows. This may be done by an accelerometer, gyroscope or other sensor. At 1002, the number of motion units of each context window may be determined. This may be done by counting the number of cycles in the motion data. At 1003, the motion unit length of each context window may be determined. This may be done by accessing a model that determines the motion unit length as a function of the number of motion units and the duration of the motion units. The motion unit length of each context window may be variable from the motion unit length of another context window. At 1004, the distance of travel of each context window may be determined. This may be done based on the number of motion units in each context window and the motion unit length of each context window. At 1005, the total distance of travel may be determined based on the distance of travel of each context window. At 1006, the total distance of travel may be presented on a user interface coupled to the wearable device. In other embodiments, the steps may be varied, and the sequence of the steps may be varied, and other processes may be performed.

FIG. 10B illustrates another exemplary process for a dynamic distance manager, according to some examples. This process may be used to determine the number of motion units of each context window. At 1011, the activity being performed by the user is determined. This may be done by the user inputting the activity he is engaged in through a user interface. This may also be done by comparing the motion data with one or more patterns saved in memory, each pattern representing an activity. For example, the process may determine that the user is running. At 1012, a nominal context window and nominal step window may be set. For example, a nominal context window may be 3 seconds and a nominal step window may be 0.5 seconds. At 1013, motion data is received. At 1014, a new context window is started. At 1015, a new step window is started. At 1016, the number of motion units is counted. This may be done by counting the number of cycles in the motion data. At 1017, the process checks whether the end of the nominal step window has passed. For example, the process determines whether 0.5 seconds has passed. If no, the process goes to 1016 and continues counting. If yes, the process goes to 1018. At 1018, the step window is adjusted to end at the transition of a motion unit closest to the end of the nominal step window. At 1019, the process checks whether the end of the nominal context window has passed. For example, the process determines whether 3 seconds has passed. If no, a new step window is started at 1015. If yes, the context window is adjusted to end at the transition of a step window closest to the end of the nominal context window. At 1021, the number of motion units of the context window is determined. This is done by adding the number of motion units of all the step windows making up the context window. At 1022, the process determines whether the activity has ended. If yes, the process ends. If no, the process goes to 1014 to start a new context window. The process continues to determine the number of motion units of each context window until the activity has ended. In other embodiments, the steps may be varied, and the sequence of the steps may be varied, and other processes may be performed.

FIG. 10C illustrates another exemplary process for a dynamic distance manager, according to some examples. This process may be used to create a model and then apply the model. At step 1031, the number of motion units for one or more samples is determined. For each sample, a person may walk a known distance. The number of motion units taken may be determined through motion data received on a sensor, or by a person counting the number of motion units. At step 1032, the motion unit length of each sample is determined. This may be done by dividing the known distance of each sample by the number of motion units of each sample. At 1033, a model determining motion unit length as a function of the number of motion units and the duration of the motion units may be created. This may be done by plotting the samples and fitting a curve or other relationship through the samples. At 1034, motion data of a user may be received. The motion data may include the number of motion units taken by the user and the duration of the motion units. At 1035, the model may be accessed to determine the motion unit length based on the number of motion units taken and the duration of the motion units. At 1036, the distance of travel may be determined based on the motion unit length and the number of motion units taken. At 1037, the execution of an operation is initiated. For example, a screen of the wearable device displays the distance traveled. As another example, the wearable device transmits data representing the distance traveled to a log of the user saved on a server. As another example, the wearable device is transitioned from a “normal” mode of operation to an “active” mode of operation if the distance of travel or speed of travel exceeds a certain threshold. A mode of operation may determine a sampling rate of one or more sensors, a clock rate for a processor of the wearable device, and other functions or features. For example, an active mode of operation may have a higher sampling rate of the sensors than a normal mode of operation. In other embodiments, the steps may be varied, and the sequence of the steps may be varied, and other processes may be performed.

FIG. 10D illustrates another exemplary process for a dynamic distance manager, according to some examples. This process may be used to calibrate a model and then apply the calibrated model. At 1041, calibration motion data is received. For example, a person may walk a known distance. Calibration motion data may be received by a sensor coupled to a wearable device of the person. At 1042, the number of calibration motion units is determined. This may be done by counting the number of cycles in the calibration motion data. At 1043, the calibration motion unit length is determined. This is done by dividing the known distance by the number of calibration motion units. At 1044, a model determining motion unit length as a function of a number of motion units and a duration of the motion units is retrieved. At 1045, the model is adjusted. This may be done by adding a new data point associated with the calibration motion unit length to the model, and re-fitting the curve or relationship to the new data. At 1046, motion data of a user is received. At 1047, the model that has been adjusted is used to determine motion unit length from the motion data. At 1048, the distance of travel is determined based on the motion unit length. At 1049, the execution of an operation is initiated. In other embodiments, the steps may be varied, and the sequence of the steps may be varied, and other processes may be performed.

FIG. 10E illustrates another exemplary process for a dynamic distance manager, according to some examples. This process may be used to confirm, verify or correct the determination of the total distance of travel. At 1051, motion data over a plurality of context windows is received. At 1052, the number of motion units of each context window is determined. At 1053, the motion unit length of each context window is determined. The motion unit length of each context window is variable from the motion unit length of another context window. At 1054, the distance of travel of each context window is determined. At 1055, the total distance of travel is determined.

At 1056, data representing a sensed distance of travel is received. In one embodiment, this may be done by a GPS of the wearable device. The GPS may detect the position of the user at regular intervals, thereby determining a path traveled by the user and the length of the path. This may also be done by a sensor for determining location within a cellular or micro-cellular network, which may or may not use GPS or other satellite constellations for fixing a position, or another location sensor. This may also be done in conjunction with map data. Map data may be stored on a server (e.g., Google Maps, Apple Maps, Mapquest, etc.) and accessed via a network (e.g., Internet, 4G, 3G, WiFi, etc.). Map data may include information indicating the distance between point A and point B, for example, the distance between the corner of 1^{st} Street and A Avenue and the corner of 2^{nd} Street and A Avenue. A GPS or other location sensor may detect that a user has traveled from the corner of 1^{st} Street and A Avenue to the corner of 2^{nd} Street and A Avenue, and map data may be used to determine the distance.

At 1057, the total distance of travel is compared with the sensed distance of travel to determine a difference. At 1058, the execution of an operation is executed if the difference is greater than a threshold. For example, the threshold may be 4 meters. If the total distance of travel determined at 1055 is 5 meters more than the sensed distance of travel determined at 1056, then the execution of an operation is performed at 1058. For example, the operation may be a display on a screen of the wearable device indicating that the difference is greater than the threshold. As another example, the operation may be a request presented on the user interface of the wearable device asking the user to calibrate the dynamic distance manager. A speaker of the wearable device may transmit an audio message to the user with the request. In another embodiment, the threshold may require that a significant difference between the sensed distance of travel and the total distance of travel occur multiple times. The threshold may be set to require multiple occurrences because the sensed distance of travel is generally less accurate than the total distance of travel. For example, the threshold may be a difference of 4 meters or more for 5 consecutive times that the user travels from the corner of 1^{st} Street and A Avenue to the corner of 2^{nd} Street and A Avenue. A memory may store a log of the difference between the sensed distance of travel and the total distance of travel. If the difference is greater than 4 meters for 5 consecutive times, then the execution of an operation is performed at 1058. In other embodiments, the steps may be varied, and the sequence of the steps may be varied, and other processes may be performed.

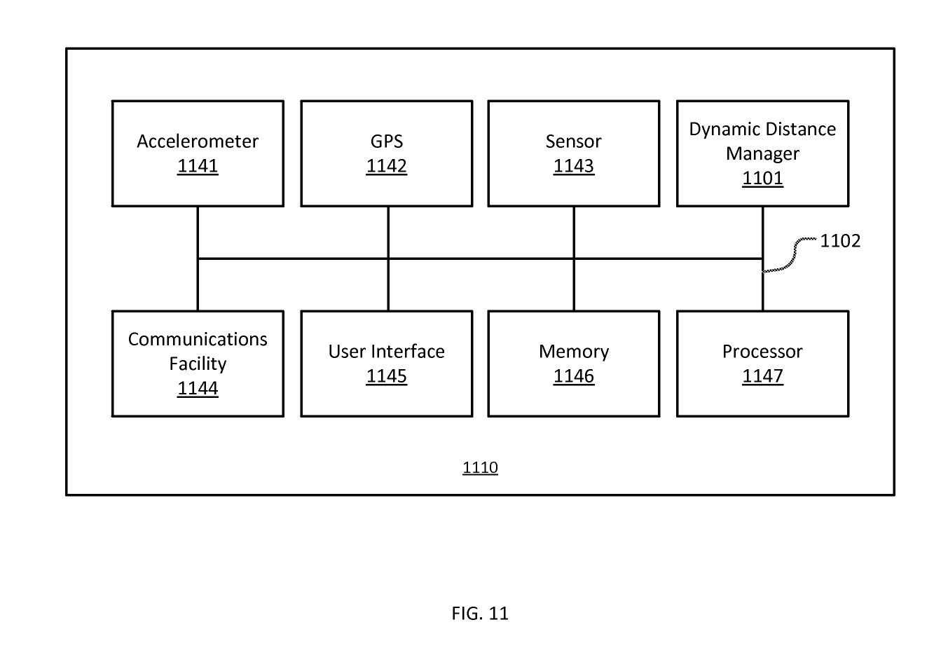

FIG. 11 illustrates a block diagram for an exemplary wearable device with a dynamic distance manager, according to some examples. Here, wearable device 1110 includes bus 1102, accelerometer 1141, GPS receiver 1142, sensor 1143, dynamic distance manager 1101, communications facility 1144, user interface 1145, memory 1146 and processor 1147. In some examples, the quantity, type, function, structure and configuration of wearable device 1110 and the elements (e.g., accelerometer 1141, GPS receiver 1142, sensor 1143, dynamic distance manager 1101, communications facility 1144, user interface 1145, memory 1146 and processor 1147) shown may be varied and are not limited to the examples provided. As shown, processor 1147 may be implemented as logic to provide control functions and signals to accelerometer 1141, GPS receiver 1142, sensor 1143, dynamic distance manager 1101, communications facility 1144, user interface 1145 and memory 1146. Processor 1147 may be implemented using any type of processor or microprocessor suitable for packaging within wearable device 1110, such as a headset, data-capable strapband, or smartphone. Data processed by processor 1147 may be stored using, for example, memory 1146. For example, one or more models may be stored in memory 1146.

In some examples, memory 1146 may be implemented using various types of data storage technologies and standards, including without limitation read-only memory (ROM), random access memory (RAM), dynamic random access memory (DRAM), static random access memory (SRAM), static/dynamic random access memory (SDRAM), magnetic random access memory (MRAM), solid state, two and three-dimensional memories, Flash® and others. Memory 1146 may also be implemented using one or more partitions that are configured for multiple types of data storage technologies to allow for non-modifiable (i.e. by a user) software to be installed (e.g., firmware installed on ROM) while also providing for storage of captured data and application using, for example, RAM. Once captured and/or stored in memory 1146, data may be subject to various operations performed by other elements of wearable device 1110.

As shown, accelerometer 1141, GPS receiver 1142 and sensor 1143 may be used as input sources for data captured by wearable device 1110. Accelerometer 1141 may gather data measured across one, two or three axes of motion. GPS receiver 1142 may be used to obtain coordinates of the geographic location of wearable device 1110 using, for example, various types of signals transmitted by civilian and/or military satellite constellations in low, medium, or high earth orbit (e.g., “LEO,” “MEO,” or “GEO”). In other examples, differential GPS algorithms may also be implemented with GPS receiver 1142, which may be used to generate more precise or accurate coordinates.

Sensor 1143 may be a location-based sensor to obtain location-based data including, but not limited to location, nearby services or items of interest, and the like. As an example, a location-based sensor may be configured to detect an electronic signal, encoded or otherwise, that provides information regarding a physical locale as wearable device 1110 passes. The electronic signal may include, in some examples, encoded data regarding the location and information associated therewith.

Still further, sensor 1143 may be implemented to provide temperature, environmental, physical, chemical, electrical or other types of sensed inputs. As presented here, sensor 1143 may include one or multiple sensors and is not intended to be limiting as to the quantity or type of sensor implemented.

Accelerometer 1141, GPS receiver 1142 and sensor 1143 may be local to or remote or distributed from wearable device 1110. Data captured by wearable device 1110 using accelerometer 1141, GPS receiver 1142 and sensor 1143 may also be exchanged, transferred or otherwise communicated through communications facility 1144. As used herein, “facility” refers to any, some or all of the features and structures that are used to implement a given set of functions. Data saved on a computer, hub or server or another wearable device (e.g., models, map data) may also be communicated through a network with communications facility 1144. For example, communications facility 1144 may include a wireless radio, control circuit or logic, antenna, transceiver, receiver, transmitter, resistors, diodes, transistors or other elements that are used to transmit or receive data to and from wearable device 1110. In some examples, communications facility 1144 may be implemented to provide a wired data communication capability such as an analog or digital attachment, plug, jack, land line or the like to allow for data to be transferred. In other examples, communications facility 1144 may be implemented to provide wireless data communication capability to transmit digitally encoded data across one or more frequencies using various types of data communication protocols, without limitation.

User interface 1145 may be implemented as a touchscreen, keyboard, mouse, joystick, LED light, display screen, vibration source, motor or other device used to serve as an interface between wearable device 1110 and the user. For example, user interface 1145 may be used to present information to the user indicating the total distance of travel. As another example, user interface 1145 may cause a vibration of a motor to signal to the user that her total distance of travel has surpassed a target. User interface 1145 may also be used to receive data manually entered by the user. The data entered using user interface 1145 may be used to specify an activity, an attribute or parameter associated with the user, the beginning or end of an activity, or a target that she wants to achieve. For example, a user may specify that she is 5.5′ tall and is about to begin swimming. The data entered using user interface 1145 may also be used to indicate that the user would like to enter the calibration mode to calibrate dynamic distance manager. In some examples, user interface 1145 may also serve as a sensor. For example, a touchscreen may be used to detect the temperature of a user's figure.

Dynamic distance manager 1101 may be used to determine a total distance of travel using the processes described above. Dynamic distance manager 1101 may be implemented or installed as part of or separate from processor 1147. Dynamic distance manager 1101 may be stored partially or wholly on memory 1146 or may be stored remotely from wearable device 1110. In still other examples, wearable device 1110 and the above-described elements may be varied in function, structure, configuration or implementation and are not limited to those shown or described.

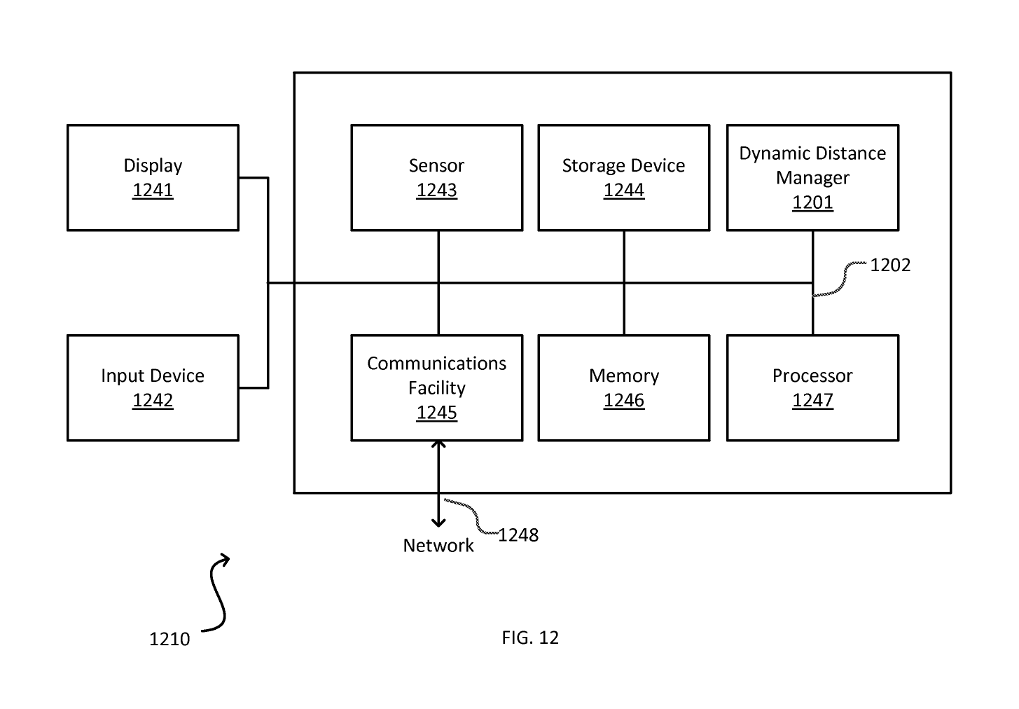

FIG. 12 illustrates an exemplary computer system suitable for use with a dynamic distance manager, according to some examples. In some examples, computer system 1210 may be used to implement computer programs, applications, methods, processes, or other software to perform the above-described techniques. Computer system 1210 may include bus 1202, display 1241, input device 1242 (e.g., keyboard, mouse or touchscreen), sensor 1243, storage device 1244, dynamic distance manager 1201, communications facility 1245 (e.g., modem or Ethernet card), memory 1246 (e.g., ROM, RAM, magnetic or optical drives) and processor 1247.

According to some examples, computer system 1210 performs specific operations by processor 1247 executing one or more sequences of one or more instructions stored in memory 1246. Such instructions may be read into memory 1246 from another computer readable medium, such as storage device 1244. In some examples, hard-wired circuitry may be used in place of or in combination with software instructions for implementation. Dynamic distance manager 1201 may be implemented as part of or separate from processor 1247, and dynamic distance manager 1201 may be stored partially or wholly on memory 1246 or storage device 1243 or another medium.

The term “computer readable medium” refers to any tangible medium that participates in providing instructions to processor 1247 for execution. Such a medium may take many forms, including but not limited to, non-volatile media and volatile media. Non-volatile media includes, for example, optical or magnetic disks. Volatile media includes dynamic memory.

Common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer can read.

Instructions may further be transmitted or received using a transmission medium. The term “transmission medium” may include any tangible or intangible medium that is capable of storing, encoding or carrying instructions for execution by the machine, and includes digital or analog communications signals or other intangible medium to facilitate communication of such instructions. Transmission media includes coaxial cables, copper wire, and fiber optics, including wires that comprise bus 1202 for transmitting a computer data signal.

In some examples, execution of the sequences of instructions may be performed by a single computer system 1210. According to some examples, two or more computer systems 1210 coupled by communication link 1248 (e.g., LAN, PSTN, or wireless network) may perform the sequence of instructions in coordination with one another. Computer system 1210 may transmit and receive messages, data, and instructions, including program, i.e., application code, through communication link 1248 and communication facility 1245. Received program code may be executed by processor 1247 as it is received, and/or stored in storage device 1244 or memory 1246, or other non-volatile storage for later execution.

Although the foregoing examples have been described in some detail for purposes of clarity of understanding, the above-described inventive techniques are not limited to the details provided. There are many alternative ways of implementing the above-described invention techniques. The disclosed examples are illustrative and not restrictive.

What is claimed:

1. A method, comprising:
receiving motion data over each of a plurality of context windows from one or more sensors coupled to a wearable device;
determining a number of motion units of each context window based on the motion data;
determining a motion unit length of each context window as a function of the number of motion units of each context window and a duration of each context window, the motion unit length of each context window being variable from the motion unit length of another context window;
determining a distance of travel of each context window based on the motion unit length of each context window;
determining a total distance of travel over the plurality of context windows based on the distance of travel over each context window; and
causing presentation of the total distance of travel on an interface coupled to the wearable device.

2. The method of claim 1, further comprising:
determining a cadence of each context window based on the number of motion units over each context window and a duration of each context window; and
determining a motion unit length of each context window as a function of the cadence of each context window.

3. The method of claim 1, wherein each context window comprises a plurality of step windows, and further comprising:
receiving motion data over each step window from the one or more sensors;
determining a number of motion units of each step window based on the motion data, the number of motion units of each step window being variable from the number of motion units of another step window;
adjusting a duration of each step window in such a way that the number of motion units of each step window is an integer, the duration of each step window being variable from the duration of another step window;
determining the number of motion units of each context window based on the number of motion units of each step window; and
determining the duration of each context window based on the duration of each step window.

4. The method of claim 3, wherein the number of motion units of each step window does not exceed three.

5. The method of claim 1, further comprising adjusting the duration of each context window in such a way that the number of motion units of each context window is an integer, the duration of each context window being variable from the duration of another context window, and each context window immediately following a context window preceding it.

6. The method of claim 1, further comprising receiving data representing one or more parameters associated with a user, and wherein the motion unit length of each context window is further determined as a function of the one or more parameters.

7. The method of claim 6, wherein the one or more parameters comprises a type of shoe being worn by the user.

8. The method of claim 6, wherein the one or more parameters comprises a physical disability of the user.

9. The method of claim 1, wherein the motion data comprises a motion vector, and further comprising:
determining a magnitude of the motion vector; and
determining the number of motion units of each context window based on a number of cycles of the magnitude over each context window.

10. The method of claim 1, wherein the motion data is associated with a swim stroke.

11. The method of claim 1, wherein the wearable device is worn by a user.

12. The method of claim 1, further comprising:
receiving data representing a target distance;
determining the total distance of travel exceeds the target distance; and
causing presentation of information indicating that the target distance is achieved on the interface.

13. A system, comprising:
a memory configured to store motion data of each of a plurality of context windows received from one or more sensors coupled to a wearable device; and
a processor configured to determine a number of motion units of each context window based on the motion data, to determine a motion unit length of each context window as a function of the number of motion units of each context window and a duration of each context window, the motion unit length of each context window being variable from the motion unit length of another context window, to determine a distance of travel of each context window based on the motion unit length of each context window, to determine a total distance of travel over the plurality of context windows based on the distance of travel over each context window, and to cause presentation of information associated with the total distance of travel on an interface coupled to the wearable device.

14. The system of claim 13, wherein the processor is further configured to determine a cadence of each context window based on the number of motion units over each context window and a duration of each context window, and to determine a motion unit length of each context window as a function of the cadence of each context window.

15. The system of claim 13, wherein the processor is further configured to adjust the duration of each context window in such a way that the number of motion units of each context window is an integer, the duration of each context window being variable from the duration of another context window, and each context window immediately following a context window preceding it.

16. The system of claim 13, wherein the processor is further configured to determine an activity associated with the motion data, to determine a caloric burn of each context window as a function of the distance of travel over each context window and the activity, to determine a total caloric burn based on the caloric burn of each context window, and to causing presentation of the total caloric burn on the interface.

17. The system of claim 13, wherein the one or more sensors comprise an accelerometer.

18. The system of claim 13, wherein the one or more sensors comprise a GPS receiver.

19. The system of claim 13, wherein the motion data is associated with an ice-skating step.

20. The system of claim 13, wherein the wearable device is carried by a user.
